(19) United States
(12) Patent Application Publication
(10) Pub. No.: US 2026/0183012 A1
(43) Pub. Date: Jul. 2, 2026

(54) SURGICAL INSTRUMENT

(71) Applicant: LIVSMED INC., Seongnam-si (KR)

(72) Inventors: Jae Hoon SHIN, Gwangju-si (KR); Young Jae SONG, Seongnam-si (KR); Gyu Dong JEON, Seoul (KR); Soon Yong JEONG, Seongnam-si (KR)

(21) Appl. No.: 19/432,599

(22) Filed: Dec. 24, 2025

(30) Foreign Application Priority Data
Dec. 31, 2024 (KR) 10-2024-0201922

(51) Int. Cl. A61B 17/29; A61B 17/00; A61B 34/37

(52) U.S. Cl. CPC A61B 17/29; A61B 34/37; A61B 2017/00199; A61B 2017/00477; A61B 2017/2929; A61B 2017/2932

(57) ABSTRACT

A surgical instrument mountable on a surgical robot includes an end tool having one or more jaws and at least one degree of rotational freedom, a wire having a first side connected to the end tool, a connection part having an inner space through which the wire passes and to which the end tool is coupled, and a driving part coupled to the connection part and configured to control rotational motion of the end tool. The driving part includes a driving pulley assembly rotatable around one axis and connected to a second side of the wire, a plate portion on which the driving pulley assembly is disposed, and a wire guide assembly coupled to the plate portion. The wire guide assembly includes a main body and at least one guide portion disposed on the main body to guide a path of the wire.

### CROSS-REFERENCE TO RELATED APPLICATION

This application is based on and claims priority under 35 U.S.C. § 119 to Korean Patent Application No. 10-2024-0201922, filed on Dec. 31, 2024, in the Ministry of Intellectual Property (MIP) of the Republic of Korea, the disclosure of which is incorporated by reference herein in its entirety.

### BACKGROUND

### 1. Field

The present disclosure relates to a surgical instrument. More particularly, the present disclosure relates to a surgical instrument that is mountable on a robot arm or manually operable for use in laparoscopic surgery or various other surgical procedures.

### 2. Description of the Related Art

Medically, surgery refers to the treatment of diseases by cutting, incising, or manipulating the skin, mucous membranes, or other tissues using medical devices. In particular, open surgery, which involves making an incision in the skin at the surgical site to treat, reconstruct, or remove internal organs, can cause bleeding, side effects, pain, scarring, and the like. Recently, surgeries performed by making a small incision in the skin and inserting only medical devices, such as laparoscopes, surgical instruments, or microsurgical microscopes, or surgeries using robots are gaining attention as alternatives.

Here, a surgical robot refers to a robot that has the function of replacing surgical actions performed by a surgeon. Surgical robots have the advantage of performing operations with greater accuracy and precision than humans and of enabling remote surgery.

Surgical robots that are currently being developed worldwide may include a bone surgical robot, a laparoscopic surgical robot, a stereotactic surgical robot, and the like. Among these, the laparoscopic surgical robot is a robot that performs minimum invasive surgery using a laparoscope and small surgical instruments.

Meanwhile, a surgical robot is generally composed of a master robot and a slave robot. When a surgical operator manipulates a control lever (e.g., a handle) provided on the master robot, a surgical tool coupled to or grasped by a robot arm equipped on the slave robot may be manipulated to perform surgery.

Laparoscopic surgery is a cutting-edge surgical technique that involves making a small incision in the navel area to insert a laparoscope, which is an endoscope used to observe the inside of the abdomen, and the technique is expected to see significant development in the future. Today's laparoscopes are equipped with computer chips and have advanced to the point where they can provide magnified images that are clearer than those seen with the naked eye, and when used in conjunction with specially designed laparoscopic surgical instruments while viewing the surgical site on a monitor, a wide range of procedures can be performed.

The background art described above is technical information retained by the present inventors in order to derive the present disclosure or obtained by the present inventors in the process of deriving the present disclosure, and thus is not necessarily known art disclosed to the general public before the filing of the present disclosure.

### SUMMARY

The present disclosure is directed to providing a surgical instrument that is mountable on a robot arm or is manually operable for use in laparoscopic surgery or various other surgeries, and that employs a wire guide assembly having an integrated structure to reduce the number of components and improve productivity.

According to an aspect of the present disclosure, a surgical instrument mountable on a surgical robot may include an end tool including one or more jaws and having at least one degree of rotational freedom, a wire having a first side connected to the end tool, a connection part configured to extend in one direction, having an inner space through which the wire passes, and having a second side to which the end tool is coupled, and a driving part coupled to a second side of the connection part and configured to control a rotational motion of the end tool. The driving part may include a driving pulley assembly configured to be rotatable around one axis and connected to a second side of the wire, a plate portion on which the driving pulley assembly is disposed, and a wire guide assembly including a main body configured to be coupled to the plate portion, and at least one guide portion disposed on the main body and configured to guide a path of the wire.

In another embodiment of the present disclosure, the at least one guide portion may redirect the path of the wire extending from the driving pulley assembly toward the inner space of the connection part.

In an embodiment of the present disclosure, the at least one guide portion may include a plurality of guide portions, and a first subset of the plurality of guide portions may be located at a first height from the plate portion while a second subset of the plurality of guide portions may be located at a second height different from the first height.

In the other embodiment of the present disclosure, the main body of the wire guide assembly may include a first body including a first guide portion, and a second body positioned on the first body and including a second guide portion.

In the other embodiment of the present disclosure, the first body and the second body may be integrally formed.

In the other embodiment of the present disclosure, the second body may be configured to move relative to the first body.

In the other embodiment of the present disclosure, the first guide portion may be located closer to the driving pulley assembly than the second guide portion.

In the other embodiment of the present disclosure, the first guide portion may be located closer to the connection part than the second guide portion.

In the other embodiment of the present disclosure, the second guide portion may space a part of the wire passing through the second guide portion apart from a part of the wire passing through the first guide portion.

In the other embodiment of the present disclosure, the first body may further include a first through-hole passing through the first body, the second body may further include a second through-hole passing through the second body, and the second through-hole may be in communication with the first through-hole.

In the other embodiment of the present disclosure, the first through-hole and the second through-hole may be in communication with the inner space of the connection part.

In the other embodiment of the present disclosure, the wire guide assembly may be disposed on an axis corresponding to a longitudinal direction of the connection part.

In the other embodiment of the present disclosure, the at least one guide portion, while being fixed to the body, may guide the path of the wire, and the wire may slidably move while being in contact with a surface of the at least one guide portion.

In the other embodiment of the present disclosure, the at least one guide portion may include a plurality of guide grooves configured to guide paths of different wires.

In the other embodiment of the present disclosure, each of the plurality of guide grooves may be recessed from a surface of the main body.

In the other embodiment of the present disclosure, the plurality of guide grooves may include at least two guide grooves that are not parallel to each other.

In the other embodiment of the present disclosure, surfaces along the paths of the plurality of guide grooves or a surface of the wire may include a friction-reducing material.

In the other embodiment of the present disclosure, each of the plurality of guide grooves may include an entry path along which the wire extending from the driving pulley assembly enters, and an exit path along which the wire passing through the entry path exits toward the connection part, and the entry path may be longer than the exit path.

In the other embodiment of the present disclosure, at least a section of each of the plurality of guide grooves may include a curved path.

In the other embodiment of the present disclosure, the wire guide assembly may further include an auxiliary guide portion configured to guide a path of the wire after the wire has passed through the guide portion.

Other aspects, features, and advantages other than those described above will become apparent from the following drawings, claims, and detailed description of the disclosure.

### BRIEF DESCRIPTION OF THE DRAWINGS

The above and other aspects, features, and advantages of certain embodiments of the disclosure will be more apparent from the following description taken in conjunction with the accompanying drawings.

FIG. 1 is a conceptual diagram illustrating a surgical robot system including a surgical robot according to an embodiment of the present disclosure.

FIG. 2 is a perspective view of the surgical robot of FIG. 1.

FIG. 3 is an enlarged perspective view illustrating some components of a first arm unit of FIG. 2.

FIG. 4 is a perspective view schematically illustrating a surgical instrument according to an embodiment of the present disclosure.

FIG. 5 is a perspective view illustrating a driving part of the surgical instrument of FIG. 4.

FIG. 6 is a perspective view illustrating the driving part of FIG. 5 with a cover removed.

FIG. 7 is a plan view of the driving part of FIG. 6.

FIG. 8 is a rear view of the driving part of FIG. 6.

FIG. 9 is a perspective view illustrating the driving part of FIG. 6, taken from a different angle.

FIG. 10 is a perspective view illustrating the driving part of FIG. 9 with wires and a middle plate removed.

FIG. 11 is a side view of the driving part of FIG. 6.

FIG. 12 is a view schematically illustrating wires connected to a wire guide assembly and a driving pulley assembly of the driving part of FIG. 6.

FIG. 13 is a perspective view illustrating the wire guide assembly of the driving part of FIG. 6.

FIG. 14 is a plan view of the wire guide assembly of FIG. 13.

FIG. 15 is a side view of the wire guide assembly of FIG. 13.

FIGS. 16 and 17 are cross-sectional views of the wire guide assembly of FIG. 13.

FIG. 18 is a plan view of a wire guide assembly according to another embodiment of the present disclosure.

FIG. 19 is a side view of a wire guide assembly according to another embodiment of the present disclosure.

### DETAILED DESCRIPTION

Hereinafter, the following embodiments will be described in detail with reference to the accompanying drawings. When describing with reference to the drawings, identical or corresponding components will be assigned the same reference numerals and duplicate descriptions thereof will be omitted.

Since various transformations can be made to these embodiments, specific embodiments will be illustrated in the drawings and described in detail in the detailed description. The effects and features of the present embodiments and the accompanying methods thereof will become apparent from the following description of the contents, taken in conjunction with the accompanying drawings. However, the present embodiments are not limited to the embodiments disclosed below, but may be implemented in various forms.

In describing the present disclosure, a detailed description of known related arts will be omitted when it is determined that the gist of the present disclosure may be unnecessarily obscured.

In the following embodiments, singular forms are intended to include plural forms as well, unless the context clearly indicates otherwise. Although terms such as “first,” “second,” and the like may be used to describe various components, such components should not be limited to the above terms. The terms are only used to distinguish one component from another.

In the following embodiments, terms such as “include” or “have” mean that the features or components described in the specification are present, and the possibility that one or more other features or components will be added is not excluded in advance.

In the following embodiments, when a unit, region, or component is referred to as being formed on another unit, region, or component, it can be directly formed on the other unit, region, or component. That is, for example, intervening units, regions, or components may be present.

In the following embodiments, terms such as “connecting” or “coupling” two members do not necessarily mean a direct and/or fixed connection or coupling of the two members, unless the context clearly indicates otherwise, and do not preclude another members from being interposed between the two members.

Sizes of components in the drawings may be exaggerated or reduced for convenience of description. For example, since the size and thickness of each component shown in the drawings are arbitrarily illustrated for convenience of description, the following embodiments are not necessarily limited thereto.

In the following embodiments, an x-axis, a y-axis, and a z-axis are not limited to three axes of the rectangular coordinate system, and may be interpreted in a broader sense. For example, the x-axis, the y-axis, and the z-axis may be perpendicular to one another, or may represent different directions that are not perpendicular to one another.

In cases where certain embodiments may be implemented otherwise, a specific process sequence may be performed differently from the described sequence. For example, two consecutively described processes may be performed substantially at the same time or performed in an order opposite to the described order.

A surgical robot 10 to which a motor pack according to an embodiment of the present disclosure is applicable will be described first.

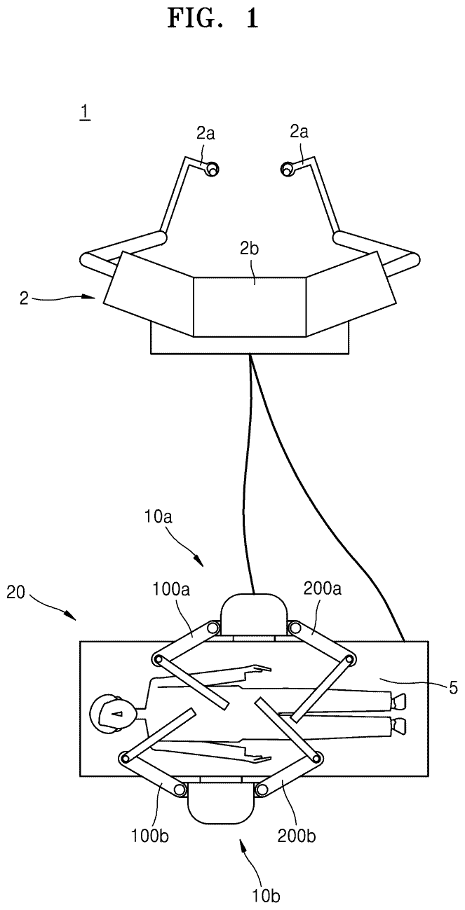

FIG. 1 is a conceptual diagram illustrating a surgical robot system including a surgical robot according to an embodiment of the present disclosure.

Referring to FIG. 1, a surgical robot system 1 includes a master robot 2 and the surgical robot 10.

The master robot 2 includes manipulating members and a display member, and the surgical robot 10 includes one or more robot arm units 100 and 200.

In one embodiment, the master robot 2 includes manipulating members 2a so that a surgical operator can grip and manipulate them respectively with both hands. In another embodiment, an image captured through a laparoscope is displayed as a screen image on a display member 2b of the master robot 2. In the other embodiment, a virtual manipulation panel may be displayed independently or together with an image captured through a laparoscope or the like on the display member 2b. A detailed description of the arrangement, configuration, and the like of such a virtual manipulation panel will be omitted.

In the other embodiment, the surgical robot 10 may include at least two robot arm units 100 and 200. Here, the robot arm units 100 and 200 may be provided in a modular form so that they can operate independently, and an algorithm for preventing collisions between the robot arm units 100 and 200 may be applied to the surgical robot system 1.

The surgical robot system 1 may include one or more surgical robots 10. FIG. 1 illustrates an embodiment in which the surgical robot system 1 includes two surgical robots 10a and 10b, each of which includes two robot arm units 100 and 200, and a total of four robot arm units 100a, 200a, 100b, and 200b are arranged accordingly.

In an embodiment, surgical instruments SI may be attached to two or more of the robot arm units 100a, 200a, 100b, and 200b, and a laparoscope may be attached to at least one of the robot arm units 100a, 200a, 100b, and 200b. In the other embodiment, a surgical operator may use the master robot 2 to select any of the robot arm units 100a, 200a, 100b, and 200b for control. As described above, by directly controlling three or more surgical instruments through the master robot 2, the surgical operator may accurately and freely manipulate various instruments on the surgical bed 5 according to the surgical operator's intent, without requiring a surgical assistant.

Hereinafter, the configuration and operating principle of the surgical robot 10 will be described in detail.

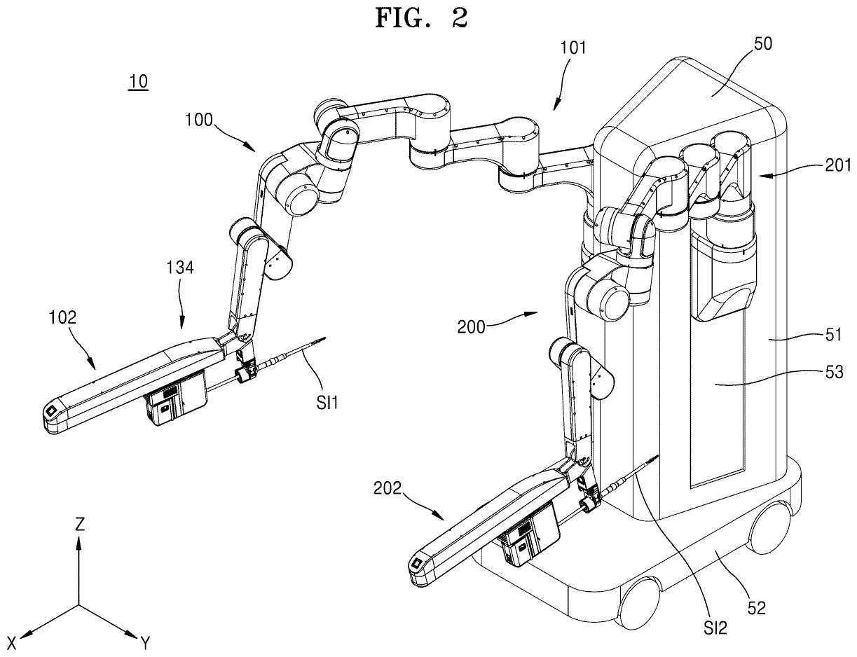

FIG. 2 is a perspective view of the surgical robot of FIG. 1.

Referring to FIG. 2, the surgical robot 10 may include a body part 50, a first arm unit 100, and a second arm unit 200. FIG. 2 illustrates an embodiment in which the surgical robot 10 includes two robot arm units, and hereinafter, the robot arm units are defined as the first arm unit 100 and the second arm unit 200, respectively.

The body part 50 functions as a body of the surgical robot 10, on which the first arm unit 100 and the second arm unit 200 may be disposed. In the other embodiment, the body part 50 may serve as a reference point for the driving of the first arm unit 100 and the second arm unit 200.

The body part 50 may include a first body 51 and a second body 52. The first arm unit 100 and the second arm unit 200 may be disposed on the first body 51, and the second body 52 may support the first body 51. In the other embodiment, the second body 52 may include wheels, as shown in FIG. 2, thereby enabling the surgical robot 10 to move.

The body part 50 may have a lifting guide 53. The lifting guide 53 may be provided in correspondence with the number of robot arm units disposed on the body part 50. The lifting guide 53 may be recessed in one side of the body part 50, and each of the robot arm units 100 and 200 may be coupled to the lifting guide 53 so as to be slidable in a first direction.

In describing the present disclosure, the portion closer to the body part 50 will be referred to as a proximal end, and the portion farther from the body part 50 will be referred to as a distal end. For example, the portion of the first arm unit 100 closer to the body part 50 is defined and described as a proximal end 101 of the first arm unit 100, and the portion of the first arm unit 100 farther from the body part 50 is defined and described as a distal end 102 of the first arm unit 100. In the other embodiment, the portion of the second arm unit 200 closer to the body part 50 is defined and described as a proximal end 201 of the second arm unit 200, and the portion of the second arm unit 200 farther from the body part 50 is defined and described as a distal end 202 of the second arm unit 200.

The first arm unit 100 may be disposed on one side of the body part 50 and may have a first surgical instrument SI1 mounted thereon. The surgical robot 10 may drive the first arm unit 100 to adjust the position and posture of the first surgical instrument SI1.

A first arm connection part (not shown) may include a plurality of connection links, and a posture thereof may be determined according to the driving of each connection link. A remote center of motion (RCM) point RCM1 of the first surgical instrument SI1 may be determined according to the posture of the first arm connection part. In this case, the RCM point of the first surgical instrument SI1 refers to an imaginary center point that serves as a rotational reference for the first surgical instrument SI1. The first surgical instrument SI1 may perform yaw and pitch motions by rotating around the RCM point.

The second arm unit 200 may be disposed on another side of the body part 50 and may have a second surgical instrument SI2 mounted thereon. The surgical robot 10 may drive the second arm unit 200 to adjust the position and posture of the second surgical instrument SI2.

A second arm connection part (not shown) may include a plurality of connection links, and a posture thereof may be determined according to the driving of each connection link. An RCM point of the second surgical instrument SI2 may be determined according to the posture of the second arm connection part. In this case, the RCM point of the second surgical instrument SI2 refers to an imaginary center point that serves as a rotational reference for the second surgical instrument SI2. The second surgical instrument SI2 may perform yaw and pitch motions by rotating around the RCM point.

Each of the arm units may include a plurality of arm connection links and a plurality of arm extension links. The arm connection links and arm extension links may rotate around their respective reference axes, and such rotational motions allow the arm unit to adjust the posture and position of the surgical instrument within an operational range.

Here, a detailed description of the arm connection links and the arm extension links will be omitted.

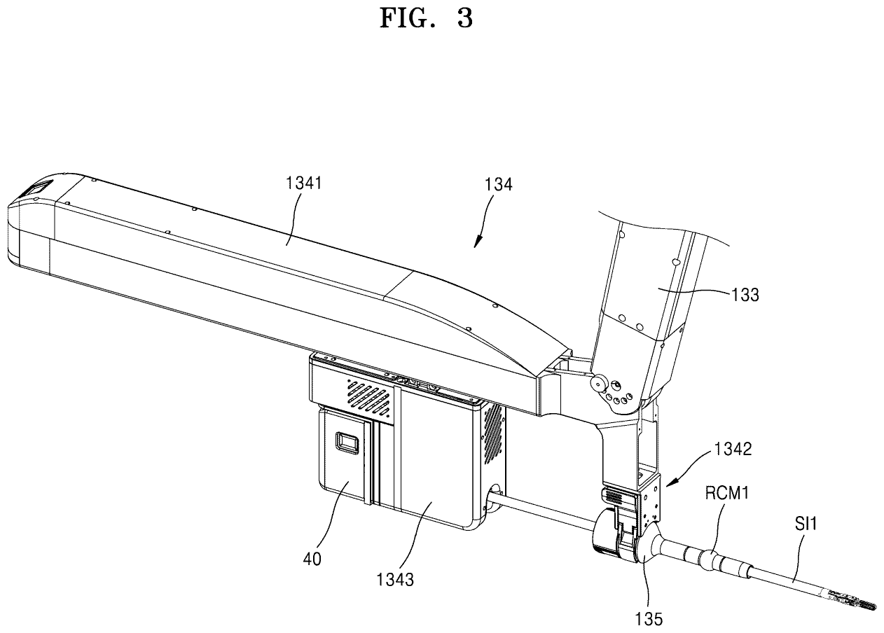

FIG. 3 is an enlarged perspective view illustrating some components of the first arm unit 100 of FIG. 2.

Referring to FIGS. 2 and 3, the first arm unit 100 according to an embodiment may include a first arm slide link 134 on the side of the distal end 102.

Here, the first arm slide link 134 may allow sliding movement of the first surgical instrument SI1.

The first arm slide link 134 may be coupled to one end, i.e., on a distal end side, of the first arm third extension link 133, and the first surgical instrument SI1 may be disposed on the first arm slide link 134.

The first arm slide link 134 may include a translation arm 1341, a slide motor pack 1343, a driving part 40, and a trocar holder 1342.

The translation arm 1341 may be coupled to one end of the first arm third extension link 133 and may move together with the first arm third extension link 133. That is, when the first arm third extension link 133 is driven, a posture of the translation arm 1341 may change accordingly.

The slide motor pack 1343 may provide a driving force for the sliding movement of the first surgical instrument SI1. The slide motor pack 1343 may include one or more first motors, as well as various components configured to generate and transmit a driving force.

The first surgical instrument SI1 may be connected to the slide motor pack 1343 through the driving part 40 and may be linearly moved by the slide motor pack 1343.

In the other embodiment, the driving part 40 may receive a driving force from the slide motor pack 1343 and transmit the driving force to an end tool of the first surgical instrument SI1.

That is, the slide motor pack 1343 may receive power from an external source to generate a driving force, and the driving force generated by the slide motor pack 1343 may be transmitted to the first surgical instrument SI1, whereby the first surgical instrument SI1 is able to perform a pitch motion, a yaw motion, an actuation motion, and a roll motion.

The trocar holder 1342 may be disposed in one region of the translation arm 1341, and a trocar 135 may be mounted to the trocar holder 1342. In the other embodiment, the trocar holder 1342 may be disposed near a distal end portion of the first arm third extension link 133.

The trocar 135 may be mountable to the trocar holder 1342, and the first surgical instrument SI1 may be coupled to the trocar 135. The first surgical instrument SI1 may be mounted through the trocar 135. In the other embodiment, the first surgical instrument SI1 may be partially supported by the trocar 135 and be slidable.

The RCM point RCM1 of the first surgical instrument SI1 may be defined on one side of the trocar 135. That is, the trocar 135 may provide the RCM point on one side, which serves as a reference point for rotational movements of the first surgical instrument SI1, including yaw and pitch motions. Once the posture of the first arm connection part is determined, a position of the RCM point defined on the trocar 135 is also determined, and the position of the RCM point may remain fixed even when the first surgical instrument SI1 moves in a sliding manner.

Hereinafter, the surgical instrument according to an embodiment of the present disclosure will be described in detail.

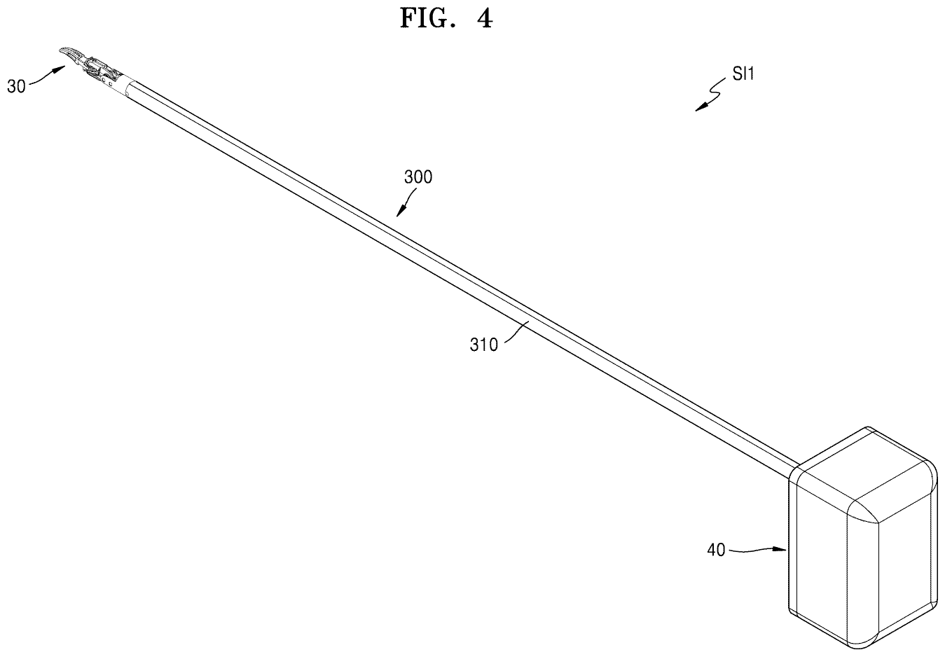

FIG. 4 is a perspective view schematically illustrating the surgical instrument according to an embodiment of the present disclosure.

The surgical instrument SI1 according to an embodiment of the present disclosure may include an end tool 30, the driving part 40, and a power transmission part 300, and the power transmission part 300 may include a connection part 310.

Here, the connection part 310 may be formed in the shape of a hollow shaft, in which one or more wires (to be described later) may be accommodated, and may have one end portion to which the driving part 40 is coupled and another end portion to which the end tool 30 is coupled, thereby serving to connect the driving part 40 to the end tool 30.

The driving part 40 may be provided at one end portion of the connection part 310 and provides an interface capable of being coupled to the robot arm unit (see 100a or the like in FIG. 1). Accordingly, when a user operates the master robot (see 2 in FIG. 1), a motor (not shown) of the robot arm unit (see 100a or the like in FIG. 1) is activated so that the end tool 30 of the surgical instrument SI1 can perform a corresponding motion, and a driving force of the motor (not shown) is transmitted to the end tool 30 via the driving part 40. In other words, the driving part 40 itself may be described as an interface that connects the surgical instrument SI1 to the surgical robot 10.

The end tool 30 may be provided at another end portion of the connection part 310, and performs necessary motions for surgery by being inserted into a surgical site. As an example of the end tool 30, a pair of jaws for performing a grip motion may be used. However, the concept of the present disclosure is not limited thereto, and various devices for performing surgery may be used as the end tool 30. For example, a configuration such as a monopolar electrocautery may also be used as the end tool, and forceps, a needle holder, a dissector, a stapler, a clip applier, and the like may also be used as the end tool. In the other embodiment, as the end tool, surgical tools such as monopolar dissectors, monopolar scissors, monopolar hooks, monopolar spatulas, bipolar dissectors, bipolar forceps, and vessel sealers may be used for electrocautery.

The above-described end tool 30 may be connected to the driving part 40 via the power transmission part 300 and receives a driving force of the driving part 40 through the power transmission part 300 to perform a motion necessary for surgery, such as a gripping motion, a cutting motion, a suturing motion, or the like.

Here, the end tool 30 of the surgical instrument according to an embodiment of the present disclosure may be configured to be rotatable in at least two directions. For example, the end tool 30 may be configured to perform a pitch motion around one rotation axis, while simultaneously performing a yaw motion and an actuation motion around another rotational axis. The end tool 30 may also be capable of performing a roll rotational motion around the connection part (shaft) as a rotation axis. In this case, the connection part 310 and the end tool 30 may perform a roll rotation together, or the end tool 30 alone may perform a roll rotation independently. In other words, the end tool may have at least one degree of rotational freedom.

Hereinafter, the driving part of the surgical instrument of FIG. 4 will be described in more detail.

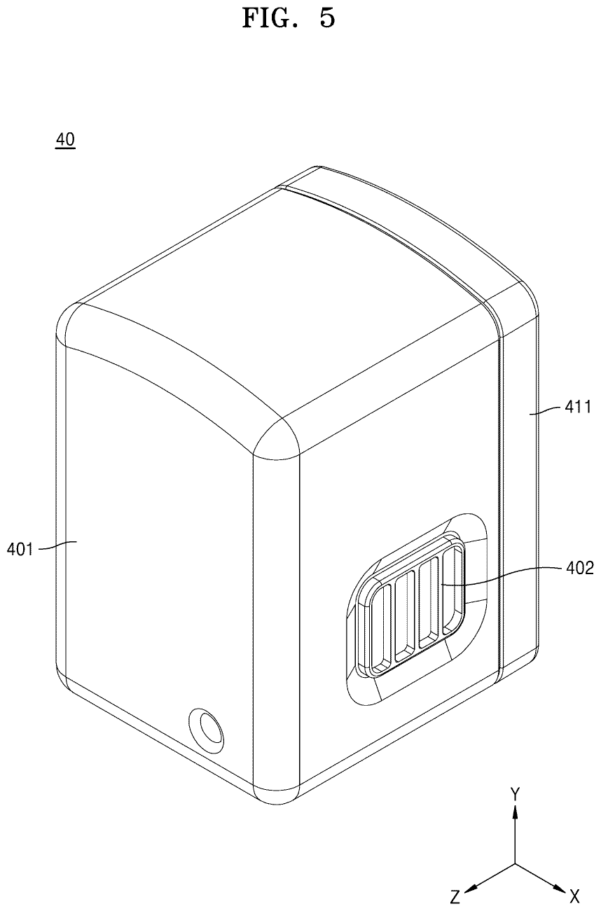

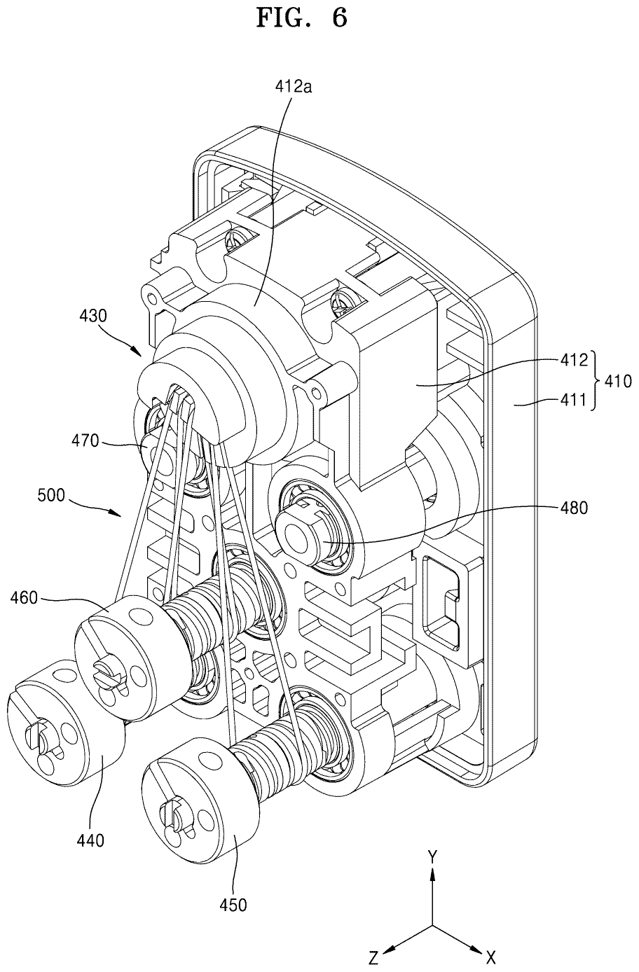

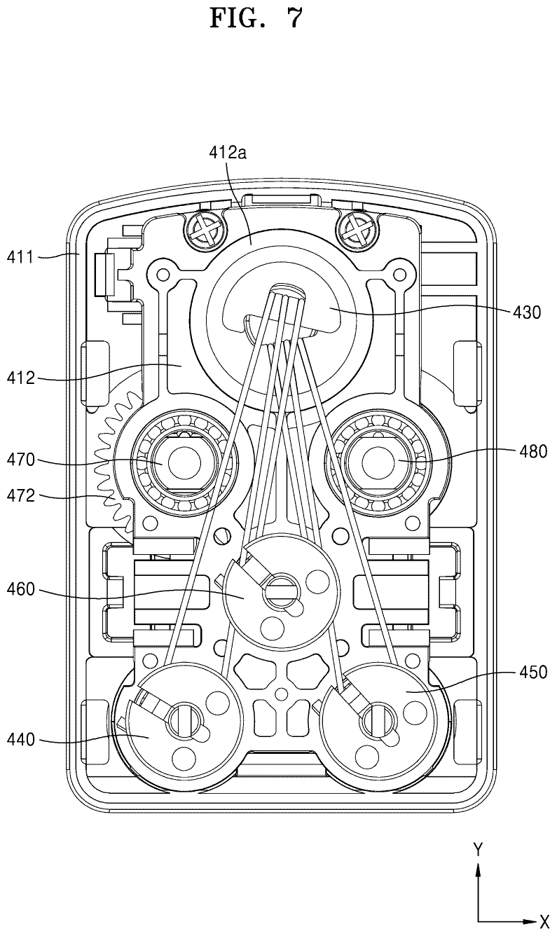

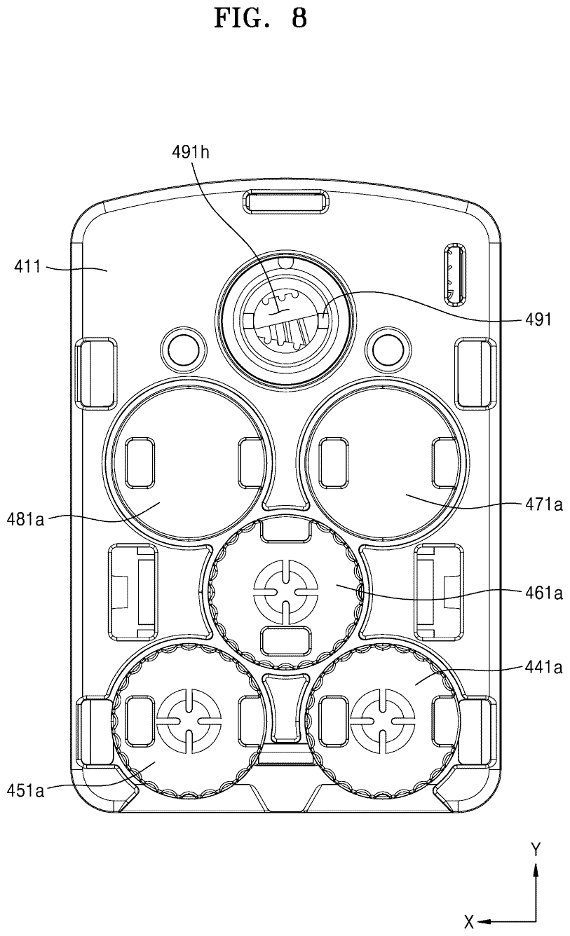

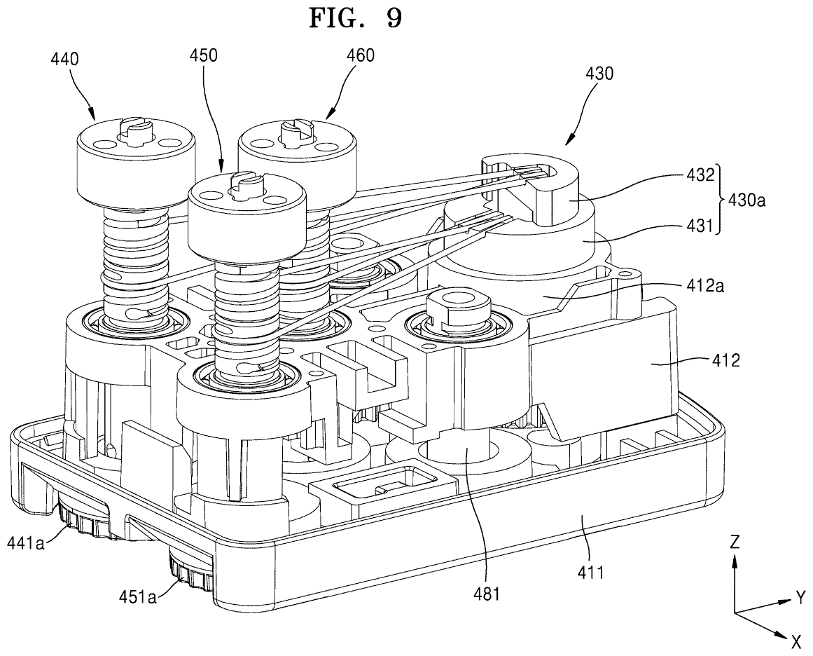

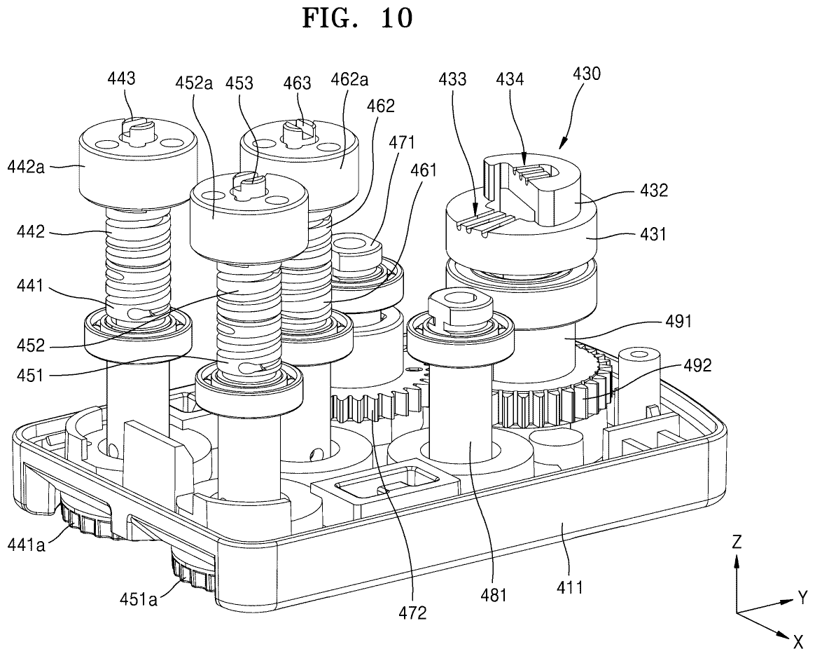

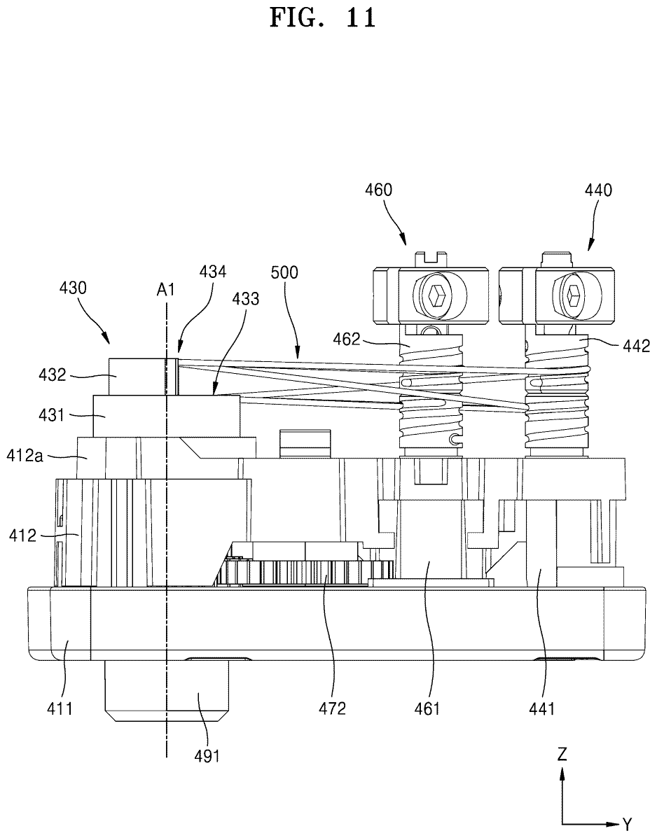

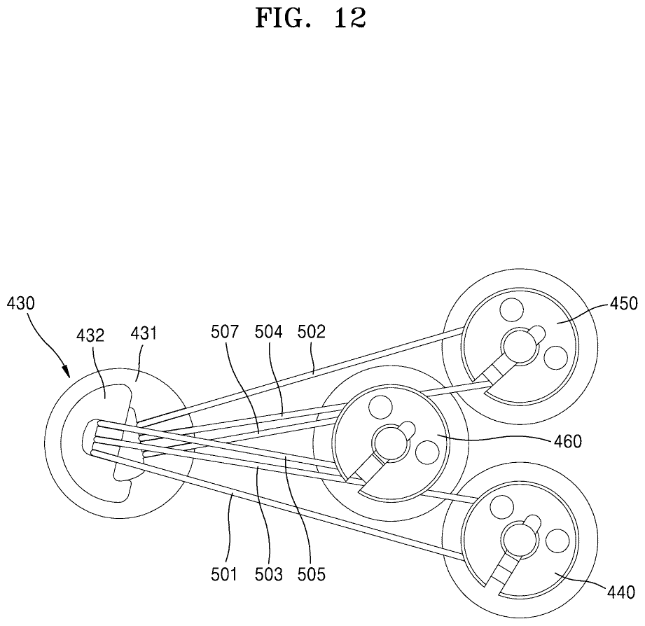

FIG. 5 is a perspective view illustrating the driving part of the surgical instrument of FIG. 4, and FIG. 6 is a perspective view illustrating the driving part of FIG. 5 with a cover removed. FIG. 7 is a plan view of the driving part of FIG. 6, and FIG. 8 is a rear view of the driving part of FIG. 6. FIG. 9 is a perspective view illustrating the driving part of FIG. 6, taken from a different angle, and FIG. 10 is a perspective view illustrating the driving part of FIG. 9 with wires and a middle plate removed. FIG. 11 is a side view of the driving part of FIG. 6, and FIG. 12 is a view schematically illustrating wires connected to a wire guide assembly and a driving pulley assembly of the driving part of FIG. 6.

Referring to FIGS. 5 to 12, the driving part 40 according to an embodiment of the present disclosure may include a plate portion 410, a cover 401, a driving pulley assembly (reference numeral not shown), and a wire guide assembly 430.

The cover 401 may be coupled to the plate portion 410 and may protect pulleys and wires inside the driving part 40. An unlock button 402 may be provided on a side surface of the cover 401. The unlock button 402 may function to decouple the driving part 40 from the slide motor pack 1343. For example, when the driving part 40 is coupled to the slide motor pack 1343, pressing the unlock button 402 may cause a locking part (not shown) to be decoupled from a coupling part (not shown) of the slide motor pack 1343, so that the driving part 40 may be brought into a removable state from the slide motor pack 1343.

The plate portion 410 may include a base plate 411 that provides a coupling surface for coupling to the above-described slide motor pack 1343, and a middle plate 412 that is disposed on a side opposite to the coupling surface of the base plate 411 and coupled to the base plate 411. That is, the base plate 411 and the middle plate 412 may be members that are separately provided and coupled to each other. However, the concept of the present disclosure is not limited thereto, and the base plate 411 and the middle plate 412 may, of course, be integrally provided.

Here, the connection part 310 having a shaft shape may be coupled to the coupling surface of the base plate 411. In the other embodiment, a shaft connector 491 may be disposed in the plate portion 410, and the shaft connector 491 may connect the above-described connection part 310 to the driving part 40. In other words, the end tool 30 may be coupled to one end portion of the connection part 310, and the shaft connector 491 may be coupled to another end portion of the connection part 310, so that the connection part 310 may be coupled to the driving part 40 through the shaft connector 491.

Here, the shaft connector 491 may serve as a passage through which wires pass after passing through the through hole of the wire guide assembly 430, which will be described later. The wires may extend into the connection part 310 through a hollow 491h of the shaft connector 491.

In the other embodiment, motor coupling parts, to which motors (not shown) for driving the driving pulleys are coupled, may be disposed on the coupling surface of the base plate 411.

Here, the motor coupling parts may be directly connected to the respective driving pulleys, or may be indirectly connected to the driving pulleys via gears.

Referring to FIG. 8, the driving part 40 according to an embodiment of the present disclosure may include a first motor coupling part 441a, a second motor coupling part 451a, a third motor coupling part 461a, a fourth motor coupling part 471a, and a fifth motor coupling part 481a. Here, the first motor coupling part 441a may function as a first jaw driving motor coupling part, the second motor coupling part 451a may function as a second jaw driving motor coupling part, the third motor coupling part 461a may function as a pitch driving motor coupling part, and the fourth motor coupling part 471a may function as a roll driving motor coupling part.

Here, each motor coupling part may be formed in the shape of a rotatable flat plate, and one or more coupling holes to which the motor (not shown) can be coupled may be formed therein.

The motors (not shown) provided in the slide motor pack 1343 may be coupled to the above-described motor coupling parts of the driving part 40, so that the driving part 40 is operated by the driving of the motors.

In the other embodiment, the middle plate 412 may provide a region in which the driving pulley assembly is disposed. For example, the middle plate 412 may provide a region in which the wire guide assembly 430 to be described later is disposed.

Referring to FIGS. 6 and 10, the driving pulley assembly may include a first driving pulley 440, a second driving pulley 450, a third driving pulley 460, a fourth driving pulley 470, and a fifth driving pulley 480.

Here, the first driving pulley 440 may be a pulley related to a rotational motion of a first jaw, and the second driving pulley 450 may be a pulley related to a rotational motion of a second jaw. In the other embodiment, the third driving pulley 460 may be a pulley related to a pitch motion of the end tool 30, and the fourth driving pulley 470 may be a pulley related to a roll rotation of the connection part 310 and the end tool 30. This will be described in detail later.

Each of the driving pulleys may be formed as a single body or may be configured with multiple parts. For example, each of the driving pulleys may include a first part, a second part, and a driving pulley rotation shaft.

In the other embodiment, the first driving pulley 440 may include a first-1 part 441, a first-2 part 442, and a first driving pulley rotation shaft 443. Here, the first-1 part 441 and the first-2 part 442 are parts to which wires are respectively connected and wound, and may be coupled to the first driving pulley rotation shaft 443 to rotate together with the first driving pulley rotation shaft 443.

The first motor coupling part 441a may be formed at one end portion of the first-1 part 441. That is, the first motor coupling part 441a may be directly coupled to the first driving pulley 440, and when the first motor coupling part 441a, which is coupled to a first jaw driving motor (not shown), rotates, the first-1 part 441, the first driving pulley rotation shaft 443, and the first-2 part 442, which are directly coupled thereto, may rotate together. That is, when the first motor coupling part 441a rotates, the first driving pulley 440 may rotate.

A groove around which a wire is wound may be formed on the side of another end portion of the first-1 part 441. That is, the groove may be formed in a portion of the first-1 part 441 adjacent to the first-2 part 442. In the other embodiment, a groove around which a wire is wound may also be formed in a portion of the first-2 part 442 adjacent to the first-1 part 441. One end portion of the first-2 part 442 may be adjacent to the first-1 part 441, and a first driving pulley head 442a may be formed at another end portion of the first-2 part 442.

In the other embodiment, the wire wound around the first-1 part 441 and the wire wound around the first-2 part 442 may be wound in opposite directions to each other. For example, the wire wound around the first-1 part 441 may be wound in a clockwise direction, and the wire wound around the first-2 part 442 may be wound in a counterclockwise direction. Accordingly, when the first driving pulley 440 rotates in one direction, one wire may be wound around the first driving pulley 440, and another wire may be unwound from the first driving pulley 440.

In the other embodiment, the second driving pulley 450 may include a second-1 part 451, a second-2 part 452, and a second driving pulley rotation shaft 453. Here, the second-1 part 451 and the second-2 part 452 are portions to which wires are respectively connected and wound, and may be coupled to the second driving pulley rotation shaft 453 to rotate together with the second driving pulley rotation shaft 453.

The second motor coupling part 451a may be formed at one end portion of the second-1 part 451. That is, the second motor coupling part 451a may be directly coupled to the second driving pulley 450, and when the second motor coupling part 451a, which is coupled to a second jaw driving motor (not shown), rotates, the second-1 part 451, the second driving pulley rotation shaft 453, and the second-2 part 452, which are directly coupled thereto, may rotate together. That is, when the second motor coupling part 451a rotates, the second driving pulley 450 may rotate.

A groove around which a wire is wound may be formed on the side of another end portion of the second-1 part 451. That is, the groove may be formed in a portion of the second-1 part 451 adjacent to the second-2 part 452. In the other embodiment, a groove around which a wire is wound may also be formed in a portion of the second-2 part 452 adjacent to the second-1 part 451. One end portion of the second-2 part 452 may be adjacent to the second-1 part 451, and a second driving pulley head 452a may be formed at another end portion of the second-2 part 452.

In the other embodiment, the wire wound around the second-1 part 451 and the wire wound around the second-2 part 452 may be wound in opposite directions to each other. For example, the wire wound around the second-1 part 451 may be wound in the clockwise direction, and the wire wound around the second-2 part 452 may be wound in the counterclockwise direction. Accordingly, when the second driving pulley 450 rotates in one direction, one wire may be wound around the second driving pulley 450, and another wire may be unwound from the second driving pulley 450.

In the other embodiment, the third driving pulley 460 may include a third-1 part 461, a third-2 part 462, and a third driving pulley rotation shaft 463. Here, the third-1 part 461 and the third-2 part 462 are portions to which wires are respectively connected and wound, and may be coupled to the third driving pulley rotation shaft 463 to rotate together with the third driving pulley rotation shaft 463.

The third motor coupling part 461a may be formed at one end portion of the third-1 part 461. That is, the third motor coupling part 461a may be directly coupled to the third driving pulley 460, and when the third motor coupling part 461a, which is coupled to a pitch driving motor (not shown), rotates, the third-1 part 461, the third driving pulley rotation shaft 463, and the third-2 part 462, which are directly coupled thereto, may rotate together. That is, when the third motor coupling part 461a rotates, the third driving pulley 460 may rotate.

A groove around which a wire is wound may be formed on the side of another end portion of the third-1 part 461. That is, the groove may be formed in a portion of the third-1 part 461 adjacent to the third-2 part 462. In the other embodiment, a groove around which a wire is wound may also be formed in a portion of the third-2 part 462 adjacent to the third-1 part 461. One end portion of the third-2 part 462 may be adjacent to the third-1 part 461, and a third driving pulley head 462a may be formed at another end portion of the third-2 part 462.

In the other embodiment, the wire wound around the third-1 part 461 and the wire wound around the third-2 part 462 may be wound in opposite directions to each other. For example, the wire wound around the third-1 part 461 may be wound in the clockwise direction, and the wire wound around the third-2 part 462 may be wound in the counterclockwise direction. Accordingly, when the third driving pulley 460 rotates in one direction, one wire may be wound around the third driving pulley 460, and another wire may be unwound from the third driving pulley 460.

A wire 501 and a wire 503, which are first jaw wires, may be connected to the first driving pulley 440. For example, the wire 501 may be wound around the first-2 part 442, and the wire 503 may be wound around the first-1 part 441. As described above, when the first driving pulley 440 rotates in one direction, the wire 501 and the wire 503 may each be wound around or unwound from the first driving pulley 440, thereby transmitting a driving force to the first jaw of the end tool 30.

A wire 502 and a wire 504, which are second jaw wires, may be connected to the second driving pulley 450. For example, the wire 502 may be wound around the second-1 part 451 and the wire 504 may be wound around the second-2 part 452. As described above, when the second driving pulley 450 rotates in one direction, the wire 502 and the wire 504 may each be wound around or unwound from the second driving pulley 450, thereby transmitting a driving force to the second jaw of the end tool 30.

A wire 505 and a wire 507, which are pitch wires, may be connected to the third driving pulley 460. For example, the wire 505 may be wound around the third-1 part 461 and the wire 507 may be wound around the third-2 part 462. As described above, when the third driving pulley 460 rotates in one direction, the wire 505 and the wire 507 may each be wound around or unwound from the third driving pulley 460, thereby transmitting a driving force to a pitch pulley of the end tool 30.

In the other embodiment, the fourth motor coupling part 471a may be formed at one end portion of the fourth driving pulley 470. That is, the fourth motor coupling part 471a may be directly coupled to the fourth driving pulley 470, and when the fourth motor coupling part 471a, which is coupled to a roll driving motor (not shown), rotates, the fourth driving pulley 470, which is directly coupled thereto, may rotate together. That is, when the fourth motor coupling part 471a rotates, the fourth driving pulley 470 may rotate.

Here, the fourth driving pulley 470 may be coupled to a roll driving gear 472. For example, the roll driving gear 472 has a structure with a central through-hole, and the fourth driving pulley 470 may be inserted into the through-hole of the roll driving gear 472 and coupled thereto. Accordingly, the fourth driving pulley 470 and the roll driving gear 472 may rotate around the same axis.

In the other embodiment, the roll driving gear 472 may be engaged with a gear 492 formed on the shaft connector 491. Accordingly, when the fourth motor coupling part 471a coupled to the roll driving motor rotates, the fourth driving pulley 470 and the roll driving gear 472 may rotate, thereby allowing the shaft connector 491 to rotate. Through this, a roll rotational motion of the connection part 310 and the end tool 30 may be controlled.

Although the fifth driving pulley 480 illustrated in the drawings is not connected to any gear device or wire, the fifth driving pulley 480 may perform a separate function by being combined with an additional component.

In an embodiment, the first driving pulley 440 has been described as a first jaw driving pulley, the second driving pulley 450 as a second jaw driving pulley, the third driving pulley 460 as a pitch driving pulley, and the fourth driving pulley 470 as a roll driving pulley. However, the concept of the present disclosure is not limited thereto, and the first driving pulley 440, the second driving pulley 450, the third driving pulley 460, the fourth driving pulley 470, and the fifth driving pulley 480 may each be formed in various positions and sizes suitable for the configuration of the driving part 40, and may perform various functions.

Hereinafter, the wire guide assembly 430 of the driving part 40 according to an embodiment of the present disclosure will be described in detail.

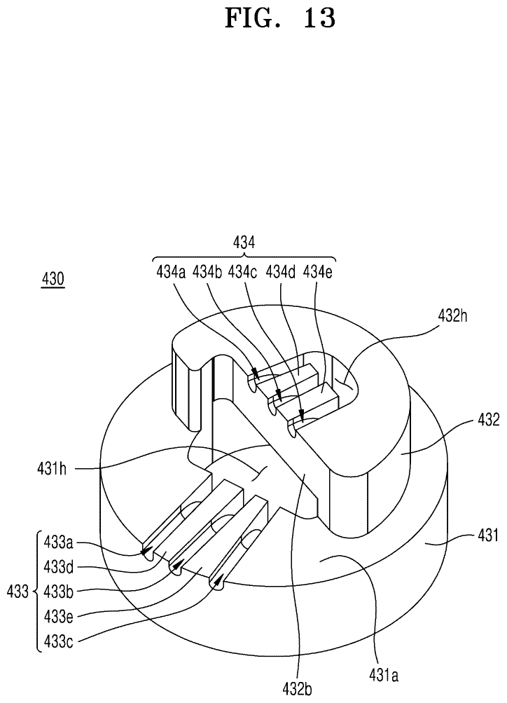

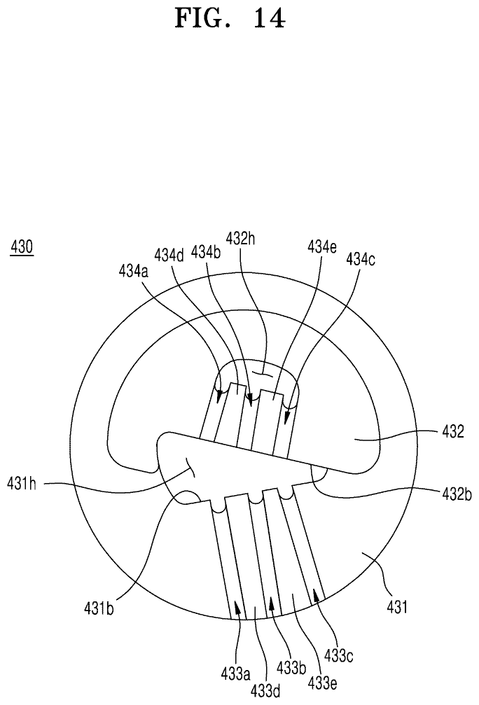

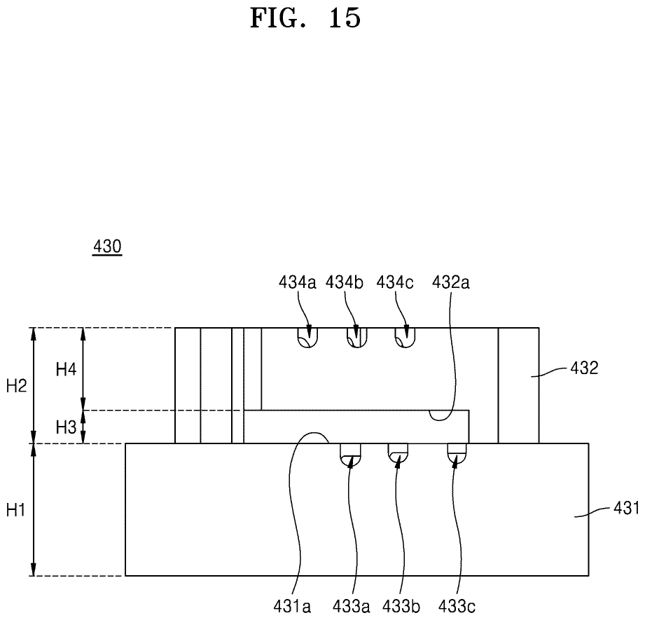

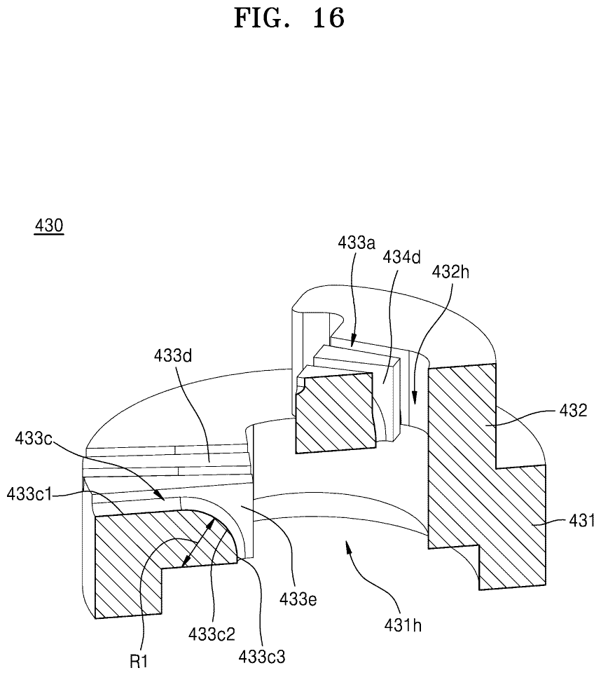

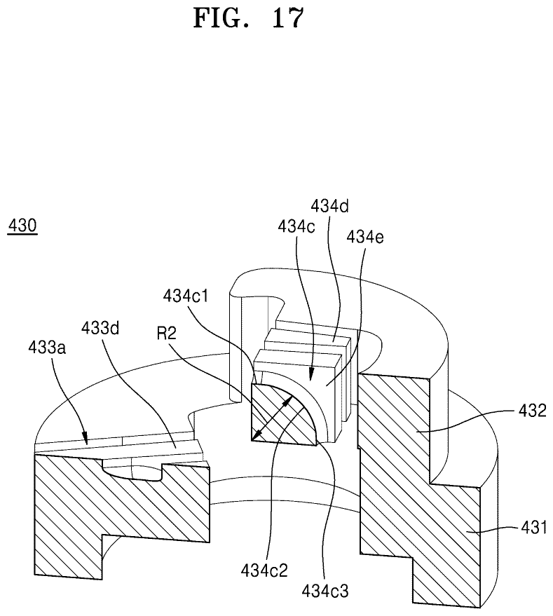

FIG. 13 is a perspective view illustrating the wire guide assembly 430 of the driving part 40 of FIG. 6, and FIG. 14 is a plan view of the wire guide assembly 430 of FIG. 13. FIG. 15 is a side view of the wire guide assembly 430 of FIG. 13, and FIGS. 16 and 17 are cross-sectional views of the wire guide assembly 430 of FIG. 13.

Referring to FIGS. 13 to 17, the wire guide assembly 430 according to an embodiment of the present disclosure may include a main body 430a that is couplable to the plate portion 410, and a guide portion that is formed on the main body 430a to guide paths of the wires.

Here, the main body 430a of the wire guide assembly 430 may be directly coupled to the above-described middle plate 412. For example, the wire guide assembly 430 may be coupled to a coupling part 412a of the middle plate 412.

Referring again to FIGS. 10 and 11, the wire guide assembly 430 may be disposed adjacent to the shaft connector 491. That is, the wire guide assembly 430 may be disposed on the side of one end portion of the shaft connector 491. For example, the wire guide assembly 430 may be disposed to be in communication with the shaft connector 491.

In other words, the wire guide assembly 430 may also be disposed to be in communication with the connection part 310. For example, the wire guide assembly 430 may be disposed on an axis corresponding to a longitudinal direction of the connection part 310.

The wires wound around the driving pulleys may extend from the driving pulleys, pass through the wire guide assembly 430, and extend into an inner space of the connection part 310. In the other embodiment, the wires may each pass through the inner space of the connection part 310 and may be connected to the pulleys of the end tool 30.

That is, the wire guide assembly 430 may be disposed such that the wires are partially in contact therewith, and may guide the wires extending from the driving pulleys to the connection part 310 by changing traveling paths of the wires.

In the driving part 40 according to an embodiment of the present disclosure, the rotation shafts of the driving pulleys are disposed parallel to the connection part 310. Since the wires wound around the driving pulleys extend in a direction parallel to the base plate 411, a component for changing the paths of the wires by approximately 90 degrees is required so that the wires can extend toward the inner space of the connection part 310.

To this end, pulleys may be disposed inside the driving part 40 to change the direction of the wires. However, because pulleys used in surgical instruments must be manufactured in extremely small sizes, it may be difficult to maintain productivity while ensuring uniform quality. This may result in increased costs and additional assembly steps, thereby making it difficult to automate the manufacturing process.

In the wire guide assembly 430 according to an embodiment of the present disclosure, by excluding a complex structure in which a micro-sized pulley is coupled to a rotation shaft, the configuration is simplified and thus the manufacturing process is streamlined, and productivity may be improved.

As such, the present disclosure may provide a surgical instrument with improved productivity by employing the wire guide assembly 430 having an integrated structure in which a complex configuration is excluded.

In the other embodiment, the main body 430a of the wire guide assembly 430 may include a first body 431 and a second body 432. Here, the first body 431 may be a portion coupled to the middle plate 412, and the second body 432 may be a portion disposed on the first body 431.

In other words, the wire guide assembly 430 may be formed in a multilayer structure including the first body 431 and the second body 432. Here, the first body 431 and the second body 432 may be integrally formed, but the concept of the present disclosure is not limited thereto.

In the other embodiment, the first body 431 may include a first guide portion 433, and the second body 432 may include a second guide portion 434 spaced apart from the first guide portion 433.

As such, the wire guide assembly 430 may include a plurality of guide portions. For example, the plurality of guide portions may be respectively formed at different positions spaced apart by varying distances from the plate portion 410. That is, when a distance from the base plate 411 to each guide portion is defined as a height of the guide portion, the plurality of guide portions may be disposed at different heights. In other words, some of the plurality of guide portions may be positioned at a first height from the plate portion, and the remaining guide portions may be positioned at a second height different from the first height.

For example, the first guide portion 433 may be disposed closer to the middle plate 412 than the second guide portion 434. In other words, the first guide portion 433 may be positioned closer to the connection part 310 than the second guide portion 434.

As such, in the wire guide assembly 430, by forming the first guide portion 433 and the second guide portion 434 at different heights, the wires passing through the second guide portion 434 and the wires passing through the first guide portion 433 can be spaced apart from each other.

In other words, the second guide portion 434 may space the wires passing through the second guide portion 434 apart from the wires passing through the first guide portion 433.

In the other embodiment, the first guide portion 433 may be formed closer to the driving pulley assembly than the second guide portion 434. In other words, the second guide portion 434 may be spaced apart from the driving pulley assembly more than the first guide portion 433.

With this structure, the wire guide assembly 430 may change the directions of the wires passing through the first guide portion 433 and the wires passing through the second guide portion 434, and gather the wires into the connection part 310 without having the wires contacting each other.

The first guide portion 433 may redirect the paths of the wires extending from the driving pulley assembly so that the wires extend toward the inner space of the connection part 310. For example, the first guide portion 433 may guide the paths of the wire 502 and the wire 504 extending from the second driving pulley 450. In the other embodiment, the first guide portion 433 may also guide the path of the wire 507 extending from the third driving pulley 460.

In a state in which the first guide portion 433 is fixed to the main body 430a, the first guide portion 433 may guide the path of each of the wire 502, the wire 504, and the wire 507 such that the wires contact a surface of the first guide portion 433 and slidably move along the surface. In other words, the first guide portion 433 does not move or rotate, and only the wires are slidably movable.

The second guide portion 434 may redirect the paths of the wires extending from the driving pulley assembly so that the wires extend toward the inner space of the connection part 310. For example, the second guide portion 434 may guide the paths of the wire 501 and the wire 503 extending from the first driving pulley 440. In the other embodiment, the second guide portion 434 may also guide the path of the wire 505 extending from the third driving pulley 460.

In a state in which the second guide portion 434 is fixed to the main body 430a, the second guide portion 434 may guide the path of each of the wire 501, the wire 503, and the wire 505 such that the wires contact a surface of the second guide portion 434 and slidably move along the surface. In other words, the second guide portion 434 does not move or rotate, and only the wires are slidably movable.

In the other embodiment, the first body 431 may include a first through-hole 431h passing through the first body 431, and the second body 432 may include a second through-hole 432h passing through the second body 432.

The first through-hole 431h may be defined adjacent to the first guide portion 433, and may be a portion through which the wires partially in contact with the first guide portion 433 pass.

The second through-hole 432h may be defined adjacent to the second guide portion 434, and may be a portion through which the wires partially in contact with the second guide portion 434 pass.

In the other embodiment, the second body 432 may be disposed on the first body 431 so as to overlap with a portion of the first through-hole 431h. In other words, the second body 432 may expose a portion of the first through-hole 431h.

In other words, in the first through-hole 431h, a portion not overlapping the second body 432 may be exposed, while a portion overlapping the second body 432 may be in communication with the second through-hole 432h. That is, the second through-hole 432h may be in communication with the first through-hole 431h.

Here, the first through-hole 431h and the second through-hole 432h may be in communication with the inner space of the connection part 310. For example, the first through-hole 431h and the second through-hole 432h may be in communication with the inner space of the connection part 310 through the shaft connector 491.

Accordingly, the wires passing through the second through-hole 432h may extend toward the connection part 310 together with the wires passing through the first through-hole 431h.

The guide portions may each be formed as a structure that includes a path along which a wire moves. For example, the guide portion may be formed in the shape of a groove or a hole to prevent the wire from deviating from the path. By way of example, the guide portion according to one embodiment of the present disclosure is described as including a guide groove, but is not necessarily limited thereto.

The guide portion may include a plurality of guide grooves configured to guide the paths of different wires. Here, each of the plurality of guide grooves may be recessed from a surface of the main body 430a. For example, the guide groove may be formed with a width corresponding to a width of the wire. In the other embodiment, widths of the guide grooves may be configured differently from each other. In the other embodiment, one guide groove may guide the path of a corresponding wire. In the other embodiment, a guide wall may be formed between adjacent guide grooves.

For example, the first guide portion 433 may include a guide groove 433a, a guide groove 433b, and a guide groove 433c. The guide groove 433a and the guide groove 433b may be separated and distinguished by a guide wall 433d, and the guide groove 433b and the guide groove 433c may be separated and distinguished by a guide wall 433e.

The second guide portion 434 may include a guide groove 434a, a guide groove 434b, and a guide groove 434c. The guide groove 434a and the guide groove 434b may be separated and distinguished by a guide wall 434d, and the guide groove 434b and the guide groove 434c may be separated and distinguished by a guide wall 434e.

For example, the guide groove 433a may guide the path of the wire 507, the guide groove 433b may guide the path of the wire 504, and the guide groove 433c may guide the path of the wire 502. In the other embodiment, the guide groove 434a may guide the path of the wire 501, the guide groove 434b may guide the path of the wire 503, and the guide groove 434c may guide the path of the wire 505. However, this is provided by way of example, and the wires disposed in the respective guide grooves may be variously modified.

Here, the plurality of guide grooves may include two or more guide grooves that are not parallel to each other.

For example, the guide groove 433a, the guide groove 433b, and the guide groove 433c may be formed parallel to each other, but may also be arranged such that adjacent guide grooves have a predetermined angle with respect to each other.

In the other embodiment, referring to FIGS. 12 and 14, the guide grooves formed in the second guide portion 434 and the guide grooves formed in the first guide portion 433 may be arranged to have a predetermined angle with respect to each other.

For example, based on an imaginary line passing through the wire guide assembly 430 and the third driving pulley 460, when the first driving pulley 440 is disposed on the left side and the second driving pulley 450 is disposed on the right side, the guide grooves of the first guide portion 433 may be formed in a direction facing the second driving pulley 450, and the guide grooves of the second guide portion 434 may be formed in a direction facing the first driving pulley 440.

The direction in which the guide grooves are formed may be configured to correspond to a direction in which the wires extend from the driving pulley. Accordingly, the direction of the guide grooves may be determined in consideration of the positions at which the first driving pulley 440, the second driving pulley 450, and the third driving pulley 460 are disposed and the arrangement of the wires wound around each of the driving pulleys.

In the other embodiment, the guide grooves formed in the first guide portion 433 may be arranged on the same plane, and the guide grooves formed in the second guide portion 434 may also be arranged on the same plane.

Accordingly, the wires positioned in the guide grooves formed in the first guide portion 433 may not interfere with each other, and the wires positioned in the guide grooves formed in the second guide portion 434 may also not interfere with each other.

Referring to FIG. 15, as described above, by arranging the second guide portion 434 and the first guide portion 433 at different heights, the paths of the wires can be three-dimensionally implemented, thereby minimizing interference between the wires positioned in the guide grooves of the first guide portion 433 and the wires positioned in the guide grooves of the second guide portion 434.

In the other embodiment, a height of the first body 431 may correspond to H1, and a height of the second body 432 may correspond to H2. The first guide portion 433 may be formed on an upper surface of the first body 431, and the second guide portion 434 may be formed on an upper surface of the second body 432. Accordingly, a height difference between the first guide portion 433 and the second guide portion 434 may correspond approximately to H2. As illustrated in the drawings, the height of the first body 431 and the height of the second body 432 may be approximately similar to each other, but are not necessarily limited thereto, and the heights of the first body 431 and the second body 432 may be variously configured.

As described above, the second body 432 may be disposed on the first body 431 and may partially overlap the first through-hole 431h. For example, a lower surface 432a of the portion of the second body 432 overlapping the first through-hole 431h may be formed to be spaced apart from an upper surface 431a of the first body 431. A spacing distance between the lower surface of the second body 432 and the upper surface of the first body 431 may correspond to H3.

The guide groove may form a path along which the wire moves. The guide groove may include an entry path and an exit path. For example, the guide groove may include an entry path along which the wire extending from the driving pulley assembly enters the guide groove, and an exit path along which the wire passing through the entry path exits toward the connection part 310. In the other embodiment, at least a section of the guide groove may include a curved path.

FIG. 16 is a view illustrating a cross-section of the wire guide assembly 430 including a cross-section of the guide groove 433c to describe a path of the guide groove 433c.

Referring again to FIGS. 13 to 16, the guide groove 433a, the guide groove 433b, and the guide groove 433c may form respective paths for the wires positioned on the first body 431.

For example, the guide groove 433c may include an entry path 433c1 and an exit path 433c3. Here, a at least a section of the guide groove 433c may be formed as a curved path 433c2. In other words, the guide groove 433c may include the entry path 433c1, the curved path 433c2, and the exit path 433c3.

In the other embodiment, the entry path 433c1, which may be formed parallel to the upper surface of the first body 431, may be where the wire first comes into contact and may be formed as a longer section than the exit path 433c3. That is, the entry path 433c1 may be formed longer than the exit path 433c3.

The curved path 433c2 of the guide groove 433c may be formed in a shape having a substantially quarter-circular cross-section. In other words, the path of the guide groove 433 c may be formed in a rounded shape on the side closer to the first through-hole 431h. That is, it may be described that a region of the guide groove 433c adjacent to the first through-hole 431h is curved to have a predetermined curvature in a cross-section.

Alternatively, from another perspective, the guide groove 433c may also be described as functioning as a type of pulley member in that the wire is wound around its outer circumferential surface, thereby guiding the path of the wire. However, the guide groove 433c is not a member that rotates around a predetermined axis like a conventional pulley, but is formed to be fixed as a part of the wire guide assembly 430. Nevertheless, the guide groove 433c may be described to partially perform a function similar to that of a pulley in that a wire is wound around the outer circumferential surface of the guide groove 433c.

Here, in the drawings, the guide groove 433c is illustrated as having a substantially quarter-circular cross-section. That is, at least a portion of the cross-section of the guide groove 433c is illustrated as having a predetermined arc shape. For example, a portion corresponding to the curved path 433c2 may be formed in a shape corresponding to a portion of a circle having a radius R1.

However, the concept of the present disclosure is not limited thereto, and the guide groove 433c may be formed to have a cross-sectional shape with a predetermined curvature, such as an ellipse or a parabola. Alternatively, the guide groove 433c may be formed in various shapes and sizes suitable for guiding the path of the wire, for example, a polygonal column having edges that are rounded to a certain extent.

Although the guide groove 433c has been described as a representative example among the guide grooves of the first guide portion 433, the shapes of the guide groove 433a and the guide groove 433b may also be similar to that of the guide groove 433c within a corresponding range.

Although the guide groove is illustrated in the drawing as being formed from the end of a side surface of the first body 431 to the first through-hole 431h, the concept of the present disclosure is not limited thereto, and the guide groove may alternatively be formed only on a portion of the upper surface of the first body 431, as needed. As such, by additionally forming guide grooves in the guide portion, unnecessary friction with the wires may be reduced, thereby improving wire durability.

FIG. 17 is a view illustrating a cross-section of the wire guide assembly 430 including a cross-section of the guide groove 434c to describe a path of the guide groove 434c.

Referring again to FIGS. 13 to 17, the guide groove 434a, the guide groove 434b, and the guide groove 434c may form respective paths for the wires disposed on the second body 432.

For example, the guide groove 434c may include an entry path 434c1 and an exit path 434c3. Here, a partial section of the guide groove 434c may be formed as a curved path 434c2. In other words, the guide groove 434c may include the entry path 434c1, the curved path 434c2, and the exit path 434c3.

In the other embodiment, unlike the guide groove 433c of the first guide portion 433, the guide groove 434c of the second guide portion 434 may be formed such that most of the path from the entry path to the exit path is a curved path.

Here, the guide groove 434c may be formed in a shape similar to that of a pulley. For example, since a wire comes into contact with only a portion of a pulley when wound around the pulley, the guide groove 434c may be formed by resembling the shape of the portion of the pulley with which the wire comes into contact. For example, the guide groove 434c may be formed in a shape resembling a pulley having a radius corresponding to a height H4 (see FIG. 15).

The curved path 434c2 of the guide groove 434c may be formed in a shape having a substantially quarter-circular cross-section. In other words, the path of the guide groove 434c may be formed in a rounded shape on the side closer to the second through-hole 432h. In other words, it may be described that a region of the guide groove 434c adjacent to the second through-hole 432h is curved to have a predetermined curvature in a cross-section.

Alternatively, from another perspective, the guide groove 434c may also be described as functioning as a type of pulley member in that the wire is wound around its outer circumferential surface, thereby guiding the path of the wire. However, the guide groove 434c is not a member that rotates around a predetermined axis like a conventional pulley, but is formed to be fixed as a part of the wire guide assembly 430. Nevertheless, the guide groove 434c may be described to partially perform a function similar to that of a pulley in that a wire is wound around the outer circumferential surface of the guide groove 434c.

Here, in the drawings, the guide groove 434c is illustrated as having a substantially quarter-circular cross-section. That is, at least a portion of the cross-section of the guide groove 434c is illustrated as having a predetermined arc shape. For example, a portion corresponding to the curved path 434c2 may be formed in a shape corresponding to a portion of a circle having a radius R2.

However, the concept of the present disclosure is not limited thereto, and the guide groove 434c may be formed to have a cross-sectional shape with a predetermined curvature, such as an ellipse or a parabola. Alternatively, the guide groove 434c may be formed in various shapes and sizes suitable for guiding the path of the wire, for example, a polygonal column having edges that are rounded to a certain extent.

In the other embodiment, surfaces along the paths of the plurality of guide grooves or surfaces of the wires may be specially treated to reduce power loss of the wires.

In the other embodiment, the surfaces along the paths of the plurality of guide grooves or the surfaces of the wires may include a friction-reducing material.

For example, special treatments for reducing friction on the surfaces along the paths of the guide grooves may include methods such as coating, heat treatment, or lubrication treatment. For example, a friction surface of the guide grooves may be treated to reduce friction through a coating method such as diamond-like carbon (DLC), Teflon, or other coatings for similar purposes. Alternatively, the friction surface of the guide grooves may be heat-treated to realize a reduction in friction. Alternatively, a lubricant may be applied to the friction surface of the guide grooves and the wire to realize a reduction in friction.

The wire guide assembly 430 according to an embodiment of the present disclosure may make it easier to ensure consistent quality compared to cases where a micro-sized pulley is used, by reducing friction between the wire and the guide portion through the use of such a friction-reducing material.

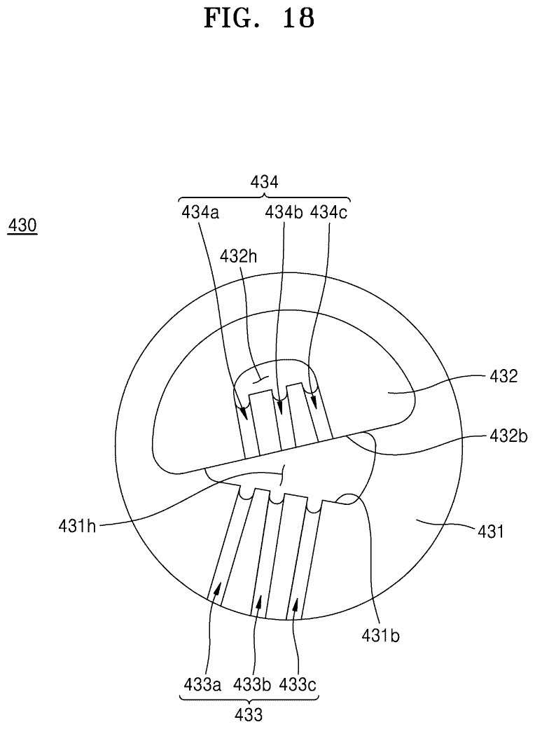

FIG. 18 is a plan view of a wire guide assembly according to another embodiment of the present disclosure.

Referring to FIG. 18, in a wire guide assembly 430 according to another embodiment, a second body 432 may be movable relative to a first body 431. In other words, the second body 432 may be configured to be rotatable on the first body 431.

For example, in the wire guide assembly 430 of FIG. 14, in a plan view, a first surface 431b of the first body 431 and a second surface 432b of the second body 432 may intersect with each other on the right side relative to the first guide portion 433, and may be spaced apart from each other on the left side. On the other hand, in the wire guide assembly 430 of FIG. 18, in a plan view, a first surface 431b of the first body 431 and a second surface 432b of the second body 432 may intersect with each other on the left side relative to the first guide portion 433, and may be spaced apart from each other on the right side.

In other words, directions in which the paths of the first guide portion 433 and the second guide portion 434 are formed may be changed. For example, the first guide portion 433 may be disposed to face the first driving pulley 440, and the second guide portion 434 may be disposed to face the second driving pulley 450.

Accordingly, the structure of a main body 430a of the wire guide assembly 430 according to another embodiment may be appropriately modified according to the positioning of the wires in the driving part 40.

The wire guide assembly 430 according to an embodiment of the present disclosure eliminates a complex structure in which a micro-sized pulley is coupled to a rotation shaft, thereby simplifying the overall configuration and reducing the number of components,. This results in a streamlined manufacturing process, and improved productivity.

As such, the present disclosure may provide a surgical instrument with improved productivity by employing the wire guide assembly having an integrated structure from which a complex configuration is excluded.

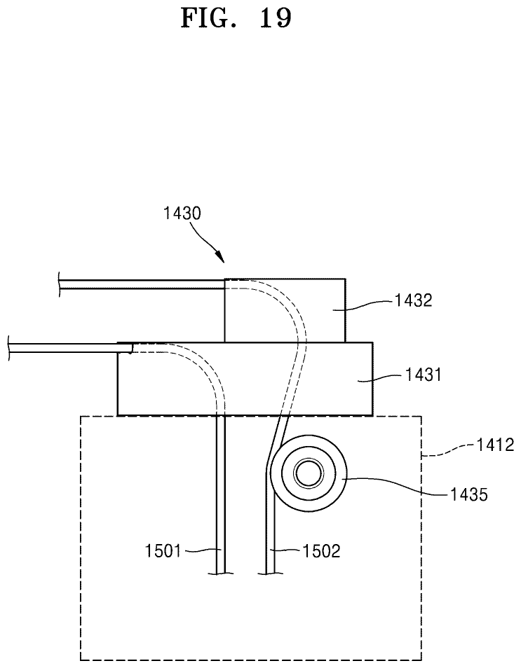

FIG. 19 is a side view of a wire guide assembly 1430 according to another embodiment of the present disclosure.

The wire guide assembly 1430 may include a first body 1431 and a second body 1432. Here, since the first body 1431 and the second body 1432 are substantially the same as the first body 431 and the second body 432 of the above-described embodiment, a detailed description thereof will be omitted.

The wire guide assembly 1430 according to another embodiment of the present disclosure may further include an auxiliary guide portion. The auxiliary guide portion may guide the path of the wire that has passed through the guide portion. Here, the auxiliary guide portion may be a pulley, but may also be a guide portion having a guide groove similar to the guide groove 433a described above. Alternatively, the auxiliary guide portion may be a guide portion having a pulley shape. That is, the auxiliary guide portion may be an additional guide portion that serves as an auxiliary pulley.

Although an auxiliary pulley 1435 is illustrated in FIG. 19, this is merely an example, and the concept of the present disclosure is not limited thereto.

In an embodiment, a wire 1502 that has passed through a second guide portion may pass through the auxiliary pulley 1435 and may be positioned to be close to a wire 1501 that has passed through a first guide portion. That is, the auxiliary pulley 1435 may change the path of the wire. Accordingly, the wires may be gathered close to each other, thereby allowing multiple wires to be guided into a narrow inner space of the connection part.

The auxiliary pulley 1435 may be disposed on a middle plate 1412. However, the position at which the auxiliary pulley 1435 is disposed, as well as the size and shape of the auxiliary pulley 1435, are not limited thereto, and the auxiliary pulley 1435 may also be formed as a portion of the main body of the wire guide assembly 1430, similar to the second guide portion.

Accordingly, the wire guide assembly 1430 according to the present embodiment may allow precise control of the wire path by including an additional structure.

According to the present disclosure, the number of components of a surgical instrument can be reduced, thereby enabling optimization of a manufacturing process and improving productivity.

The present disclosure has been described above with reference to exemplary embodiments. It will be understood by those skilled in the art that various modifications and changes in form and details may be made thereto without departing from the essential features of the present disclosure. Therefore, the disclosed embodiments should be considered in a descriptive sense and not for purposes of limitation. The scope of the present disclosure is defined not by the detailed description of the disclosure but by the appended claims, and all differences within the scope will be construed as being included in the present disclosure.

What is claimed is:

1. A surgical instrument mountable on a surgical robot, the surgical instrument comprising:
an end tool including one or more jaws and having at least one degree of rotational freedom;
a wire having a first side connected to the end tool;
a connection part configured to extend in one direction, having an inner space through which the wire passes, and having a first side to which the end tool is coupled; and
a driving part coupled to a second side of the connection part and configured to control a rotational motion of the end tool, the driving part including:
a driving pulley assembly configured to be rotatable around one axis and connected to a second side of the wire;
a plate portion on which the driving pulley assembly is disposed; and
a wire guide assembly including a main body configured to be coupled to the plate portion, and at least one guide portion disposed on the main body and configured to guide a path of the wire.

2. The surgical instrument of claim 1, wherein the at least one guide portion redirects the path of the wire extending from the driving pulley assembly toward the inner space of the connection part.

3. The surgical instrument of claim 1, wherein
the at least one guide portion includes a plurality of guide portions,
a first subset of the plurality of guide portions are located at a first height from the plate portion, and
a second subset of the plurality of guide portions are located at a second height different from the first height.

4. The surgical instrument of claim 1, wherein the main body of the wire guide assembly includes:
a first body including a first guide portion; and
a second body disposed on the first body and including a second guide portion.

5. The surgical instrument of claim 4, wherein the first body and the second body are integrally formed.

6. The surgical instrument of claim 4, wherein the second body is configured to move relative to the first body.

7. The surgical instrument of claim 4, wherein the first guide portion is located closer to the driving pulley assembly than the second guide portion.

8. The surgical instrument of claim 4, wherein the first guide portion is located closer to the connection part than the second guide portion.

9. The surgical instrument of claim 4, wherein the second guide portion spaces a part of the wire passing through the second guide portion apart from a part of the wire passing through the first guide portion.

10. The surgical instrument of claim 4, wherein
the first body further includes a first through-hole passing through the first body,
the second body further includes a second through-hole passing through the second body, and
the second through-hole is in communication with the first through-hole.

11. The surgical instrument of claim 10, wherein the first through-hole and the second through-hole are in communication with the inner space of the connection part.

12. The surgical instrument of claim 1, wherein the wire guide assembly is disposed on an axis corresponding to a longitudinal direction of the connection part.

13. The surgical instrument of claim 1, wherein the at least one guide portion, while being fixed to the main body, guides the path of the wire, and the wire slidably moves while being in contact with a surface of the at least one guide portion.

14. The surgical instrument of claim 1, wherein the at least one guide portion includes a plurality of guide grooves configured to guide paths of different wires.

15. The surgical instrument of claim 14, wherein each of the plurality of guide grooves is recessed from a surface of the main body.

16. The surgical instrument of claim 14, wherein the plurality of guide grooves include at least two guide grooves that are not parallel to each other.

17. The surgical instrument of claim 14, wherein surfaces along the paths of the plurality of guide grooves or a surface of the wire include a friction-reducing material.

18. The surgical instrument of claim 14, wherein
each of the plurality of guide grooves includes an entry path along which the wire extending from the driving pulley assembly enters, and an exit path along which the wire passing through the entry path exits toward the connection part, and
the entry path is longer than the exit path.

19. The surgical instrument of claim 18, wherein at least a section of each of the plurality of guide grooves includes a curved path.

20. The surgical instrument of claim 1, wherein the wire guide assembly further includes an auxiliary guide portion configured to guide a path of the wire that has passed through the at least one guide portion.
